(19) United States
(12) Patent Application Publication
(10) Pub. No.: US 2004/0071860 A1
(43) Pub. Date: Apr. 15, 2004

(54) Process for producing a multiplicity of building blocks of a library of materials

(76) Inventors: John Michael Newsam, San Diego, CA (US); Stephan Andreas Schunk, Heidelberg (DE); Jens Klein, Heidelberg (DE)

Correspondence Address: Stephen D Scanlon, North Point, 901 Lakeside Avenue, Cleveland, OH, 44114, US

(21) Appl. No.: 10/433,496

(22) PCT Filed: Nov. 30, 2001
(86) PCT No.: PCT/EP01/14045

(30) Foreign Application Priority Data
Dec. 1, 2000 (DE) 100 59 890.0

(51) Int. Cl. A61L 2/00

(52) U.S. Cl. 427/2.1

(57) ABSTRACT

The process of the invention permits the synthesis of large libraries of materials of multicomponent systems, in which all mixtures of binary to polynary systems are synthesized rapidly and arbitrarily in the multicomponent system. Combining an inventive library of materials with an inventive test for performance characteristic extremely accelerates the screening of a large number of novel materials.

The present invention relates to a process for producing building blocks of a library of materials according to the preamble of Claims 1, 4, 17 and 20, and to a library of materials which is obtainable by an inventive process, and to a process for identifying various building blocks in a library of materials according to the preamble of Claim 32 and to a process for determining performance characteristics and/or properties of building blocks or materials in a library of materials according to the preamble of Claim 36.

The present invention is in the field of combinatorial chemistry, in particular the field of producing and testing libraries of materials in the search for performance characteristics of constituents of such libraries of materials. This technical field is described intensively both in the patent literature and in scientific publications.

Within a few years, combinatorial synthesis has developed into an important method in the research of active compounds in the field of pharmaceutical chemistry. In addition to the conventional procedure of synthesizing one substance after another, increasing importance is being gained by methods which are able to prepare simultaneously many substances of defined structure. A combinatorial synthesis is characterized in that, in one synthesis step, a reaction is carried out not with only one synthesis building block, but with many in parallel or in a mixture. In each step, all possible combinations are performed so that with only a few building blocks, a great number of products, a “library of materials”, is formed. Such syntheses of libraries of compounds have been known hitherto in the areas of peptide chemistry and biochemistry. This leads to libraries which extend from a few individual compounds to many thousands of substances in mixtures. Thus the spatially separated synthesis of chain-like materials on two-dimensional supports is routinely used in biochemistry and peptide chemistry (G. Jung, A. G. Beck-Sickinger, Angewandte Chemie 1992, 104, pp. 375-391). By means of a combination of solid-phase synthesis and photolithography, it is possible to apply in a spatially separated manner not only photolabile protecting groups, which can be destroyed by irradiation, but also to couple amino acids or ligands successively. The position of each peptide can be established by the masking technique strategy.

In combinatorial peptide chemistry, in addition, what is termed “split and pool” synthesis is widely used and is the subject of numerous publications (F. Balkenhohl et al., Angew. Chemie 1996, 108, 2436-2488, G. Lowe, Chem. Soc. Rev. 1995, 24, 309-317, G. Jung. A. G. Beck-Sickinger, Angew. Chemie 1992, 104, 375-391, M. C. Pirrung, Chem. Rev. 1997, 97, 473-488, U.S. Pat. No. 5,556,762). The basis of this synthesis technique is the Merrifield solid-phase synthesis. In this method, for example, functionalized polystyrene beads are used as solid-phase support. In a first step, for example, a great number of polystyrene beads are split among three containers and an amino acid A, B and C is coupled to each of the three subsets. Then the three subsets are recombined (pool) and mixed. This operation is repeated, for example, twice more, so that at the end 27 different permutations of tripeptides have been rapidly and simultaneously synthesized. The purpose of such a procedure can be seen from the fact that when the 20 proteogenic amino acids are used 400 (20²) different dipeptides, 8000 (20³) different tripeptides, 160 000 (20⁴) different tetrapeptides, 32 000 000 (20⁵) different pentapeptides, etc., may be synthesized. In addition, it is an advantage that the synthesis occurs via successive coupling of covalent bonds and the quantitative yield of the reactions is unambiguous, as a result of which defined molecular units can be synthesized on a defined number of polymer beads.

Further, WO 91/04266 and U.S. Pat. No. 5,160,378 disclose using chemically modified ends of polymer rods for combinatorial synthesis. The peptide synthesis takes place via anchor groups in a defined manner at the end of these rods, the arrangement of which permits the use of conventional microtitre plate formats for synthesis or wash steps by immersion in reagent-filled cavities.

In inorganic chemistry, to date, combinatorial techniques have only been disclosed in the field of two-dimensional arrays.

Thus U.S. Pat. No. 5,985,356 describes the synthesis of materials by coating and testing of two-dimensional material arrays on an inert substrate. By using sputter techniques and using of microstructured masks, whose dimensions in the micrometre range can only be achieved by lithographic techniques, large material yields having a multiplicity of building blocks may be produced in a very small space completely automatically. By successive use and rotation of the masks, differing components can be deposited on defined regions. As a result of the temperature treatment after the deposition, material systems are formed by interdiffusion between the approximately 100 nm thick layers. By using automated systems, these material arrays can be produced repeatedly; in the case of differing conditions of the secondary treatment steps, the effect of the secondary treatment parameters on the formation of new phases and materials can also be controlled (U.S. Pat. No. 6,004,617). Disadvantages are the restriction to a two-dimensional support, the use of small amounts of materials in the microgram range, the bonding to defined support materials, the extremely small dimensions of the individual material position and the lack of control over the material's morphology.

WO 00/17413 describes the successive addition of suspensions or solutions of individual components to defined regions of a support together with post-conditioning steps, as a result of which direct, spatially resolved, two-dimensional library production is possible. The support material is divided into regions by physical barriers (for example spotting plate array or depressions/boreholes on any desired support). All of these said processes for producing inorganic materials by the route of combinatorial chemistry, however, share the feature that the synthesis is performed on rigid or semi-rigid two-dimensional predefined surfaces and that the number of materials produced is a direct function of the working steps, or dispensing steps or deposition steps associated with the production.

The object of the present invention was therefore to extend the techniques known in combinatorial chemistry to provide a process for producing multicomponent systems with components that are not tied to a two-dimensional substrate comprising materials, such as catalysts, that may, in fact, be produced as unsupported bulk-phase bodies. Only in three-dimensional embodiments is it possible to create reaction zones or diffusion zones or concentration gradients or any combination thereof and to thereby control the activity or selectivity of the catalyst body. Obviously, this is not possible for homogeneous films or multilayers on a substrate. A further object of the present invention is to establish a process for testing materials of such a library of materials, with which it is possible to accelerate and optimize the synthesis and characterization of libraries of materials in comparison with the abovementioned processes and apparatuses, and thus to obtain libraries of materials which are further improved with respect to the rapidity and bandwidth of the synthesis of multicomponent systems and using which libraries having up to more than 10⁶ building blocks can be produced in a very efficient manner.

These and other objects are achieved by the inventive process for producing a multiplicity of building blocks of a library of materials, in which the inventive process comprises a sequence of the following steps:

(2) contacting all or parts of a number t₁ₘ of support bodies of the kind T1 of at least one subset M₁ₘ with at least one first substance of the kind S₁ₛ, characterized in that the support bodies are porous and that at least parts of the substance penetrates into at least parts of the bulk of the support bodies, and where s, t₁, and m are mutually independent integers ≧1;

(3) combining parts of or complete subsets M₁ₘ to result in one or more second sets M₂ₙ containing a number t₂ₙ of support bodies of the kind T1, where n and t₂ are integers ≧1 and n<m.

In a preferred embodiment, the process has the following further step (1), which more preferably is carried out before steps (2) and (3):

(1) dividing a first set M₁ containing a number t₁ of support bodies of the kind T1 into a number k of first subsets M₁ₘ containing a number t₁ₘ of support bodies, where m and t₁ independently of one another are integers ≧1, and t₁≧k with k being an integer ≧2.

In a further preferred aspect, the inventive process comprises the further step (0) in which the first set M₁ of the number t₁ of support bodies of the kind T1 is contacted before step (1) with at least one substance zero of the kind S₀ₛ, where s has the above meaning.

A further advantageous embodiment of the inventive process comprises a sequence of the following steps:

(I) contacting at least one first substance of the kind S₁ₛ with a first set M₁ containing a number t₁ of support bodies of the kind T1;

(1) dividing a first set M₁ of the number t₁ of support bodies of the kind T1 into a number k of first subsets M₁ₘ containing a number t₁ₘ of support bodies of the kind T1, where m and t₁, are mutually independent and integers ≧1, and t₁≧k with k being an integer >2.

Preferably, in the inventive process, the first set M₁ of t₁ support bodies comprises at least two different support bodies T1X, where X is an integer ≧2.

Further preference is given to a sequence of the following steps:

(2)-(3)-(I)-(1);

(3)-(2)-(I)-(1);

(2)-(I)-(3)-(1); or

(3)-(1)-(I)-(2),

where before and/or after and/or during and/or instead of at least one of steps (I) and (0) to (3) at least one physical and/or chemical and/or physicochemical treatment is carried out on at least one of the support bodies T1, T1X and T2Y, in which case the respective treatment of the individual support bodies can be identical or different.

Thus, advantageously, a process is provided which permits the rapid and planned efficient synthesis of libraries of materials, with the libraries of materials produced according to the invention generally being markedly greater than the libraries produced with the previous methods and being able readily to contain 10⁹ to 10¹⁵ different building blocks.

The support bodies are preferably subjected before and/or after and/or during and/or instead of the above-defined steps (I) and (0) to (3) to at least one physical and/or chemical treatment and/or physicochemical treatment in one or more treatment steps. More preferably, only a single support body or a defined number of support bodies is treated in this manner. If a plurality of, or all, support bodies are treated in this manner, the respective treatment of the support bodies can be identical or different.

The support bodies can also originate from other sequences carried out independently or in parallel according to the inventive process.

In addition, the invention relates to a process of the type in question here, in which identical or different support bodies T2Y, which are identical to or different from the support bodies T1 or T1X, where Y is an integer ≧1, are added to at least one of the subsets M₁ₘ before and/or after and/or during the respective steps (I), (0), (1), (2) or (3) or the totality of the steps (I) and (0) to (3) or sequence of steps.

Furthermore, a freely selectable number of support bodies T1 and/or T1X and/or T2Y support bodies and/or building blocks can be taken off from and/or added to at least one of the subsets M₁ₘ before and/or after and/or during the steps (I), (0), (1), (2) and/or (3).

By means of the inventive process, the building blocks, preferably three-dimensional bodies, can be varied to contain materials selected from the following group: heterogeneous or heterogenized catalysts, luminophores, thermoelectric, piezoelectric, semiconducting, electrooptical, superconducting or magnetic substances or mixtures of two or more of these substances, in particular intermetallic compounds, oxides, nitrides, carbides, oxide mixtures, mixed oxides, ionic or covalent compounds of metals and/or non-metals, metal alloys, ceramics, active carbon, organometallic compounds and composite materials, dielectrics, thermoelectrics, magnetoresistive and magnetooptical materials, organic compounds, polymers, enzymes and enzyme mixtures, active pharmaceutical compounds, substances for feed and feed supplements, substances for foods and food supplements and cosmetics and mixtures of two or more oxides. The materials can be varied as desired, for example with respect to their stoichiometry, and then the building blocks or, for example, their stoichiometry, most suited to the respective use can be found. It is also possible that via a suitable different element composition, all variants can be tested of a multiplicity of building blocks, for example catalysts, which, although they are substantially similar, differ in their elements by at least one element.

Preferably, the subsets and/or sets produced in the context of the inventive process are the same size or different sizes. This leads to the fact that differing amounts of different catalyst variants and materials can be produced in a simple manner.

In particular, it is preferred that the building blocks are inorganic materials, since in the field of inorganic catalyst research, to date actual combinatorial synthesis methods have not been able to be used satisfactorily.

It is preferred that the support bodies are porous bodies. Such porous bodies can have micropores, mesopores, macropores according to the IUPAC definition or a combination of two or more thereof, in which case the pore distribution can be monomodal, bimodal or multimodal. Preferably, the bodies have a multimodal pore distribution having a high proportion, that is to say more than 50%, of macropores. Porous bodies or materials for such bodies which may be mentioned are: foamed ceramics, metallic foams, metallic or ceramic monoliths, hydrogels, polymers, in particular PU foams, polymer beads, in particular superabsorbers (acrylates etc.), composites, sintered glasses or sintered ceramics. Solid or porous bodies, for example metal bodies, ceramics, glasses, plastics, composites, which are provided with an appropriate pore structure by suitable processes, can also be used. Such processes can be: drilling processes, cutting processes, erosion processes, etching processes, laser lithography processes or screen printing processes.

Suitable bodies have a BET surface area of 1 to 1 000 m²/g, preferably 2 to 800 m²/g, and in particular 3 to 100 m²/g.

In addition, in the context of the inventive process, the support bodies used can be produced per se during a treatment step, for example starting from a suitable precursor, for example a powder, by means of a suitable process, for example disintegration, sol-gel processes, precipitation, melting and solidification, spraying and coating. This treatment step is preferably carried out before the start of the actual process, that is to say before step (I), (0), (1) or (2).

In a further embodiment, the support bodies can be altered during any of the steps mentioned above or in a pretreatment before any of the steps mentioned above or in a working-up procedure occurring after completion of the steps mentioned above. The term “altered” as used in the present invention relates to any physical or chemical or physical-chemical change, including the formation of covalent or ionic bonds, or any combination thereof, that the support bodies are subjected to, including but not limited to a complete or partial reaction of the support body material with any or all of the substances that it is brought in contact with during any of the steps mentioned above, a complete or partial intermixing of the support body material with any or all of the substances that it is brought in contact with, as well as a complete or partial removal of the support body material.

The substances can be applied to the support bodies in dissolved, suspended, emulsified, dispersed or molten form. The substances can be brought into contact with each suitable solvent and then applied, for example, by impregnation, spraying, sponging or immersion. Further processes for applying the substances are powder coating processes and methods for applying microencapsulated substances.

An additional degree of freedom can be generated by inhomogeneous application of substances, for example in the form of a solution, to a set or subset of support bodies. In this embodiment, the solution volume applied is less than the liquid absorption capacity of the support bodies. As a result, advantageously not all support bodies are uniformly impregnated with the solution. Therefore, gradients of the respective substance are generated on each application step.

In addition, it is possible to achieve an inhomogeneous application of substances by covering some of the set of support bodies, for example, and thus contacting only a subgroup with the substance. For the contacting, local spray systems, for example, are suitable, preferably with a mask or diaphragm to cover the remaining support bodies.

The present invention thus also relates to a process which comprises a sequence of the following steps:

(1′) contacting a first subgroup of sets and/or subsets of support bodies with at least one first zero substance S₀ₛ and/or at least one first substance S₁ₛ,where s is an integer and can be identical or different for each step that is repeated;

(2′) mixing with one another respective sets and/or subsets contacted according to (1′), and

(3′) contacting a second subgroup of sets or subsets of support bodies with at least one second substance S₂ₛ which is identical to or different from the substances S₁ₛ and/or S₀ₛ and where s can be identical or different for each step that is repeated.

In each generation of support bodies, in accordance with the above embodiment, support bodies can be present without this substance and those which bear exclusively one substance can be present.

Preferably, the support volume, that is to say the fluid absorption capacity of the support bodies, is adjustable. This can be adjusted, for example, via conditioning with an inert gas, vapor or a liquid for example water vapor. Thus the absorption capacity of each support body can be adjusted exactly, so that only a maximum loading with a substance solution, which loading can be set in advance, is possible.

It is advantageous if the support bodies, after each application of substances, are redistributed (mix-split). Thus, in the context of the repeated sequences according to the inventive process, all polynary systems, that is to say binary, ternary, quaternary and higher systems, may be produced within a single rapid combinatorial synthesis.

Preferably, the sequence of the inventive process is repeated and/or permuted as often as desired, so that highly complex polynary systems are thus also accessible in a simple manner. In particular, it is further preferred, that after each step of application of substances, a treatment step, for example a drying step, follows. As a result, in a simple manner, the utilization of 100% of the support volume is made possible in each application.

It is also preferred that after at least one step of the sequence or of the repeated sequences a precipitant or a mineralizing agent is added, so that in this manner coprecipitation on or in the support bodies can be carried out. Suitable precipitants can be: inorganic or organic bases and/or acids. Suitable mineralizers are, for example, halides.

It is further preferred that the applied substances react with one another before, during, or after each application step, or else not until after all application steps, and thus complex polynary systems of differing stoichiometric composition are thus possible with subsequent variation of their chemical and/or physical properties.

It is further preferred that the support bodies are functionalized. Such functionalizations can change the physicochemical characteristics of the support body. Such characteristics can be: polarity, acidity, basicity, steric characteristics, complexing characteristics, electronic and ionic characteristics and the pore structure. Via a functionalization as desired, for example by applying organic adhesion promoters or compounds which make possible improved solubility of applied substances, as many substances as desired which differ in their physical characteristics can be applied, for example hydrophobic and hydrophilic substances, lipophilic and lipophobic substances. Suitable processes for this are all processes known to those skilled in the art. By this means, for example, by applying a substance, a further substance, which owing to the functionalization, is already applied, can be dissolved out by the applied substance or can react with the applied substance and thus in a simple manner even more complex precursor compounds can be applied to the support body.

In a further embodiment of the present invention, instead of support bodies contacted with substances, solutions, suspensions, emulsions, dispersions or melts of substances are employed each of which are situated inside of support bodies, for example hollow bodies, vessels, for example vials, which are suitable for receiving such fluid systems and containing them as such.

In addition, the object of the present invention is achieved by a process which comprises a sequence of the following steps:

(2) introducing at least one first substance S₁ₛ into all or into some of k partial volumes V₁ₘ, where m and s are integers ≧1 and k is an integer ≧2;

(3) combining parts of the partial volumes, or complete partial volumes V₁ₘ to give a plurality of second volumes V₂ₙ, where n is an integer and n<m.

Preferably, the process comprises a further step (1) which is further preferably carried out before the steps (2) and (3):

(1) dividing a volume V₁ of a fluid medium FM₁ into k first partial volumes V₁ₘ, where m is an integer ≧1 and k is an integer ≧2.

Further preferably, the inventive process comprises the further step (0), in which a first volume V₁ is contacted before step (1) with at least one zero substance S₀₂, where s has the above meaning.

In a further advantageous embodiment, the inventive process comprises a sequence of the following steps:

(I)introducing at least one first substance S₁ₛ into a first volume V₁;

(O)dividing the first volume V₁ of a fluid medium into k first partial volume V₁ₘ, where m is an integer ≧1 and k is an integer ≧2, further preferably a sequence of the following steps:

(2)-(3)-(I)-(1);

(3)-(2)-(I)-(1);

(2)-(I)-(3)-(1); or

(3)-(1)-(I)-(2).

Preferably, a freely selectable number of subdivided volumes UV₁ₘ is taken off from at least one of the partial volumes V₁ₘ before and/or after and/or during steps (I) and (0) to (3).

It is advantageous if before and/or after and/or during and/or instead of steps (I) and (0) to (3) at least one physical and/or chemical or physicochemical treatment is carried out on at least one of the partial volumes V₁ₘ and/or subdivided volumes UV₁ₘ, in which case the respective treatment of the individual partial volumes and/or subdivided volumes can be identical or different.

In a further preferred embodiment, the inventive process comprises a sequence of the following steps:

(1′) introducing the at least one first zero substance S₀ₛ and/or at least one first substance S₁ₛ into a first partial volume V₁ₘ and/or subdivided volume UV₁ₘ;

(2′) interchanging with one another the respective partial volume and/or subdivided volume obtained according to (1′) and

(3′) introducing at least one second substance S₂ₛ which is identical to or different from the substances S₁ₛ and/or S₀ₛ into a second subdivided volume of the volumes or partial volumes.

Thus, in a very simple manner, by mixing as desired fluid systems of different substances, as described above, which is particularly easily carried out, differing building blocks are obtained which can only be obtained at the end of the inventive process or else after each individual division step.

Advantageously, a further embodiment enables an agent to be added to the partial volumes V₁ₘ or subdivided volumes UV₁ₘ, which can be the same size or different sizes, which agent enables at least part of the dissolved substances to be obtained in solid form, particularly preferably these agents being precipitation agents or support bodies.

If support bodies as defined above are used, as a result of coprecipitation on or in the porous beads, the desired polynary material systems are formed. However, it is also preferred that precipitation reagents are added. Thus, triggering of the resultant materials can be avoided, so that by adding precipitation reagents, solid body materials are prepared by coprecipitation.

It is preferred that the sequence of the abovementioned steps is repeated and/or permuted as often as desired. By this means, highly complex polynary material systems can be obtained particularly simply. It is preferred that after the end of each sequence the precipitated substances are isolated. By this means, in a very simple manner, in the context of the repeatability, which can be carried out as frequently as desired, of the inventive sequence, the desired polynary material systems can be obtained at each step of the process.

It is preferred that the precipitated substances are subjected to a reactive treatment. This can be, for example, a thermal treatment or radiation treatment, so that even after obtaining the materials further modifications can be performed. Further possible reactive treatment methods are: spraying, spray-drying, concentration by evaporation, kneading and other physicochemical treatment methods and combinations thereof.

In a further preferred embodiment of the inventive process, it is possible that the abovementioned agent or agents are added before one or more steps of combining the subdivided solutions. By this means all conceivable material combinations in the synthesis can be obtained after each individual step.

In addition, the present invention relates to the library of materials per se which is available by the above-described processes.

A further object of the present invention is achieved by a process for identifying different building blocks of a library of materials by each building block being provided with an individualizable coding. Since the present invention is based on a rapid and arbitrary synthesis of multicomponent systems which have a great multiplicity with respect to element composition and element distribution, it is important that at least individual generations are labeled during synthesis.

It is preferred that the coding is substantially chemically inert and is not damaged by any process step. This can be achieved, for example, by adding, in varying amounts, one or more elements which are inert with respect to the process steps. By detection of this specific element after the test, unambiguous identification of the material is then possible. It is of importance in this “chemical encoding” that the component(s) should not be volatile. Therefore, for example, halogens, arsenic, selenium, tellurium, cadmium, lead or mercury are only of limited suitability.

Particularly suitable and preferred is encoding by radioactivity or gamma radiation in which the corresponding elements are introduced into the material system at each generation. By detection of the radiation using a suitable detector, such library components of this generation can be identified. Further preference is given to encoding via nanomaterials, for example chemically inert cluster compounds, for example rare earth oxides, TiO₂, ZrO₂, which have, for example, fluorescence activity or phosphorescence activity.

Examples of physicochemical measurement methods for decoding the information are X-ray diffraction, X-ray fluorescence, measurement of high-energy radiation, for example radioactivity, measurement of optical characteristics, for example absorption, fluorescence or phosphorescence characteristics.

Preferably, the methods are selected from:

RFA, UV/VIS spectroscopy, detection of high-energy radiation, NMR spectroscopy, ESR spectroscopy, Mossbauer spectroscopy, SIMS and optical methods.

In addition, the coding is preferably based on a characteristic of the support body.

The object underlying the present invention is further achieved by a process for determining physicochemical characteristics of building blocks of the inventive library of materials, which process comprises the following steps:

(a) determination of at least one performance characteristic and/or property of at least one building block using at least one sensor and if appropriate

(b) determination of at least one further performance characteristic and/or property of the at least one building block using at least one further sensor.

Preferably, the second parameter is only determined for the building blocks in the library of materials for which the measurements of the first parameter have already given an indication of a desired performance characteristic and/or property.

Further preferably, the building blocks are selected automatically for further measurement by a data processing system.

Preferably, according to the invention, building blocks or materials which occur in the solid state and are potentially suitable as heterogeneous catalysts are prepared, and if appropriate tested with respect to their performance characteristics. Thus these materials are heterogeneous catalysts and/or their precursors, further preferably inorganic heterogeneous catalysts and/or their precursors, and in particular solid catalysts or supported catalysts and/or their precursors.

In the context of the present process, the individual materials can be identical or different from one another.

Firstly, if necessary, a part of the library of materials, for example in the case of a catalyst, can be activated. This can be carried out by thermal treatment under inert gases or reactive gases or other physical and/or chemical treatments. The building blocks are then brought to a desired reaction temperature and then a fluid starting material, which can be an individual compound or a mixture of two or more compounds, is passed through or along one, a plurality or all of the parts of the library of materials.

The fluid starting material which consists of one or more reactants is generally liquid, or preferably gaseous. A mixture of liquid and gaseous phases is conceivable as well. Preferably, testing of, for example oxidation catalysts, is performed by contacting, in parallel or sequentially, individual, a plurality, or all, sections of the library of materials with a gas mixture of one or more saturated, unsaturated or polyunsaturated organic starting materials. Those which may be mentioned here are, for example, hydrocarbons, alcohols, aldehydes etc., and oxygen-containing gases, for example air, O₂, N₂O, NO, NO₂, O₃ and/or, for example, hydrogen. Furthermore, an inert gas, for example nitrogen or a noble gas can also be present. The reactions are generally carried out at temperatures from 20 to 1200° C., preferably at 50 to 800° C., and in particular at 80 to 600° C., in which case the separate removal, which is carried out in parallel or successively, of the respective gas streams of the individual parts, a plurality of parts or all parts is ensured by means of a suitable device.

Thus the present invention relates to a process in which, before step b), a starting material is introduced into at least two parts of the library of materials which are separated from one another to carry out a chemical and/or physical reaction in the presence of at least one material of the respective part and, after flow through the section, an effluent stream is obtained.

The resultant effluent stream comprises at least one reaction product which is then collected either from individual sections or a plurality of sections of the library of materials and is preferably analyzed separately, successively or preferably in parallel, if analysis of the exhaust stream is required after the inventive process in the respective part.

A plurality of reactions, each interrupted by a purge step with a purge gas can also be carried out and analyzed successively at the same or different temperatures. Obviously, identical reactions at different temperatures are also possible.

Preferably, at the start of the process, the collected effluent stream of the entire library is analyzed in order to establish whether a reaction has taken place at all.

In this manner, groups of building blocks may be analyzed very rapidly to establish whether they have any useful characteristics, for example catalytic characteristics, at all. Obviously, after carrying out this coarse screening, again individual groups of building blocks can be analyzed together, in order again to establish which group of building blocks, if a plurality of such groups of building blocks are present in the library of materials, have catalytic characteristics.

The present invention permits automated production and catalytic testing for the purpose of high throughput and screening of, for example, heterogeneous catalysts for chemical reactions, in particular for reactions in the gas phase, very particularly for partial oxidations of hydrocarbons in the gas phase with molecular oxygen (gas phase oxidations).

Reactions and conversions suitable for study are described in G. Ertl, H. Knötzinger, J. Waldkamp (editors): “Handbook of heterogeneous catalysis”, Wiley V C H, Weinheim, 1997. Examples of suitable reactions are principally listed in this reference in Volumes 4 and 5 and in Numbers 1 to 4.

The effluent lines of the effluent streams of the respectively selected sections comprise at least one reaction product and/or the starting material which is preferably obtained separately from the respective sections. This is preferably achieved via a device which is connected gas-tightly to the respective sections. In particular, devices which may be mentioned are: sample removal using suitable flow guides, for example valve circuits and mobile capillary systems (sniffing apparatuses). In a particularly preferred embodiment, sniffing apparatuses are used which have a spatially localized, for example point-shaped, heat source. This together with the fact that the heat source is coupled to the sniffing apparatus permits selective heating of the part of the library of materials under test and allows a reaction to be initiated only in this region. The individual effluent streams of the individual sections, a plurality of sections or all sections can be taken off separately here and then analyzed separately via a valve circuit.

The mechanically movable sniffing apparatus, which is, for example, computer-controlled, comprises a sniffing line or sniffing capillary to the effluent stream to be taken off which is essentially automatically positioned on, in and/or above the exit of the respective section and then takes off the effluent stream. Details with respect to the disposition of such an apparatus may be taken from WO 99/41005.

In principle there is freedom in the choice of the measurement method, but it should be in this case a relatively rapid and simple measurement method, since a great number of sections are to be analyzed. The purpose of this first measurement is a preferred preselection of those parts which must be analyzed further.

In particular, sensors which may be mentioned are: infrared thermography, infrared thermography in combination with mass spectrometry, mass spectrometry, GC, LC, HPLC, micro GC, dispersive FTIR spectroscopy, microwave spectroscopy, Raman spectroscopy, NIR spectroscopy, UV spectroscopy, UV-VIS spectroscopy, NMR spectroscopy, ESR spectroscopy, microwave spectroscopy, GC-MS, infrared thermography/Raman spectroscopy, infrared thermography/dispersive FTIR spectroscopy, color detection using a chemical indicator/MS, color detection using a chemical indicator/GCMS, color detection using a chemical indicator/dispersive FTIR spectroscopy, photoacoustic analysis, chemical or electrochemical sensors and tomographic NMR and ESR methods.

Particular preference is given to mass spectrometry and measurement methods coupled to it, and to tomographic NMR and ESR methods, optionally using specific probe molecules.

Further preferably, infrared thermography is used, which may be simply implemented using an infrared camera. In this case the temperature development of the individual parts may be taken from the recorded infrared image, preferably using digital image processing. In the case of a small number of parts, if appropriate, a temperature sensor can be assigned to each individual part, for example a pyrometric element or a thermocouple. The results of the temperature measurement for the respective distances can all be sent to a data processing system which preferably controls the inventive process. Further details of this method may be taken from WO 99/34206 and DE-A-100 12 847.5, whose contents in this respect are completely incorporated by reference in the context of the present application.

In order substantially to eliminate interfering influences, the parts containing the sections to be tested should preferably be situated in a thermally insulated housing having a controlled atmosphere. If an infrared camera is used, this should preferably be situated outside the housing, with observation of the building blocks being made possible by infrared-transparent windows, for example made of sapphire, zinc sulphite, barium difluoride, sodium chloride, etc. On the basis of the results of the measurement of the first parameter, using a data processing system or a computer, the sections are chosen for which at least one further performance characteristic can be measured. In this case, also, differing selection criteria are conceivable. Firstly, those sections can be selected for which the first parameter is better than a predefined limit value, secondly, a predefined percentage of the best of all sections or materials of a support body can alternatively be selected for measurement of a second parameter. The said minimum requirements and the amount of sections to be selected depends firstly on the respective quality requirements of the materials to be tested and secondly on the time which is available for testing the support materials.

If a limit value is preset with respect to the minimum requirement of the first measured value, this need not be constant for all parts, but it can be preset, for example, as a function of other characteristics of the respective building blocks of the individual parts of the library of materials.

The at least one further parameter (performance characteristics and/or property) is preferably measured at the effluent stream of the selected sections. In principle, the further sensor is subject to no restriction, provided that it is suitable for measuring a further parameter which gives indications of a further characteristic of the building block under test.

In principle, as further sensor, a process mentioned above for the first sensor can be employed, in which case the first and further sensor are identical to or different from one another, preferably different from one another. Preferably, this further sensor is based on a spectroscopic method which is selected from the group consisting of mass spectrometry, gas chromatography, GCMS spectroscopy, Raman spectroscopy, infrared spectroscopy, UV-VIS spectroscopy, NMR spectroscopy, fluorescence spectroscopy, ESR spectroscopy, NMR tomography and ESR tomography and Mossbauer spectroscopy.

On the basis of these preferred techniques, more precise information on the effluent stream of the respective sections or building blocks may be obtained. Using these spectroscopic methods, the concentration of a sought-after product or the concentration of parallel products and the residual concentration of the starting materials can be determined, from which, for example, for catalytic building blocks, information on selectivity may be derived.

For mass spectroscopy, preferably a quadrupole mass spectrometer is used, although TOF mass spectrometers are conceivable. The effluent stream of the sections under test is fed to the mass spectrometer or other sensors preferably via a pipe system. Further mass spectrometric arrangements which can be used are MS-MS couplings.

Preferably, the analysis is controlled by a data processing system, so that suitable parts of the library of materials can be identified particularly simply and rapidly. It is advantageously possible here to analyze specifically one constituent of a single section, since in the case of such abovementioned measurement methods, specific selection of a small region from a larger region is possible.

In addition, the invention relates to a computer program having program code means for carrying out the inventive process, and to a data carrier comprising such computer programs.

The terms used in the context of the present invention shall be explained at this point:

| | |
|---|---|
| Contacting: | In the context of the present invention, contacting is |
| | taken to mean that a substance is brought into direct |
| | contact with at least one porous support body |
| | leading to at least one of the following steps: |
| | diffusion or capillary action of the substance into |
| | the porous body, reaction of the substance with at |
| | least parts of the support body, transport within the |
| | porous body, including mass transport, physical |
| | alteration of the support body or of the substance or |
| | of both, for example by swelling or altering the pore |
| | structure |
| Building block: | Unit which is a member of a library of materials and |
| | which can consist of one or more components or |
| | materials. |
| Building block | The term “building block composition” used |
| composition: | according to the invention comprises the |
| | stoichiometric and the element composition of the |
| | materials under test conditions which can differ |
| | from material to material, as well as the spatial |
| | distribution of substances within a building block. |
| | Thus it is possible according to the invention to |
| | produce and test libraries of materials which consist |
| | of materials in which, although they are identical |
| | with respect to their element composition, the |
| | stoichiometric composition of the elements making |
| | up the material differ between the individual |
| | materials; in addition, it is possible that the library |
| | of materials is made up of materials which are each |
| | different with respect to their element composition; |
| | obviously it is also possible for the individual |
| | materials to differ in their stoichiometric and |
| | element composition; in addition it is possible that |
| | the library of materials is made up of building |
| | blocks which are identical with respect to their |
| | element composition and stoichiometric |
| | composition, but differ with respect to the physical |
| | or chemical or physicochemical characteristics as a |
| | consequence of a treatment step. The term |
| | “element” used here relates in this case to elements |
| | of the Periodic Table of the Elements. |
| Treatment step: | Physical or chemical or physical and chemical |
| | treatment of at least a part of the inventively used |
| | building blocks, materials, substances or support |
| | bodies. In this case a set of support bodies can also |
| | be treated in such a manner that the abovementioned |
| | treatments are not applied uniformly over all |
| | support bodies. Thus it is possible, for example, that |
| | a portion of the set of the support bodies is |
| | subjected to a chemical treatment, while another |
| | portion of the set of support bodies is subjected to a |
| | physical treatment, without the other portion in each |
| | case “seeing” the other treatment. In addition, it is |
| | possible to establish a gradient of the respective |
| | treatment over the set of support bodies, for |
| | example to carry out a thermal treatment in a |
| | tubular kiln, at one end of which a temperature of, |
| | for example, 500° C. is applied and at the other end |
| | of which a temperature of, for example, 1000° C. is |
| | applied, the temperature changing from one end to |
| | the other continuously or stepwise. Examples of |
| | physical treatments are treatments involving |
| | temperature, pressure or light, and examples of |
| | chemical treatments are contacting with reactive |
| | gases, for example hydrogen, ammonia, hydrochloric |
| | acid, reaction solutions, for example |
| | precipitants, mineralizing agents, adhesion |
| | promoters, binders and hydrophobicizing agents, |
| | examples of physicochemical treatments are |
| | contacting with, for example, water vapour or |
| | treatment with reactive gases with simultaneous |
| | irradiation with light. |
| Characteristics of | Characteristics of the support bodies can be, for |
| the support | example: shape, density, porosity, surface |
| bodies: | properties, plasticity, chemical composition, size, |
| | morphology. |
| Property: | The term “property” designates physical, chemical |
| | or physicochemical states of the individual materials |
| | within the library of materials; examples which may |
| | be mentioned here are: oxidation state, crystallinity, |
| | etc. |
| Introduction: | This term designates any type of feed of (chemical) |
| | substances in volumes, for example pouring, |
| | spraying, etc. |
| Fluid: | A fluid is defined as a medium whose flowability is |
| | proportional to the expression e^{-Δ E/RT}, where ΔE is |
| | the energy which must be overcome in order for the |
| | medium to flow. These include, for example, |
| | liquids, gases, waxes, dispersions, fats, suspensions, |
| | melts, pulverulent solids etc. If the medium is |
| | present in liquid form, this is also taken to mean |
| | multiphase liquid systems. |
| Coding: | Coding, in the context of the present invention, |
| | means that a physical and/or chemical characteristic |
| | of the support body is accessible to measurement. |
| | Examples of such characteristics which may be |
| | mentioned are: shape, density, porosity, surface |
| | properties, plasticity, elasticity, chemical |
| | characteristics, composition, concentration, optical |
| | and electronic characteristics. |
| Material: | Preferably non-gaseous substances, for example |
| | solids, oxides, salts, sols, gels, waxy substances or |
| | substance mixtures, dispersions, emulsions, |
| | suspensions and solids. These can be molecular and |
| | nonmolecular compounds, formulations, mixtures, |
| | where the term “nonmolecular” defines substances |
| | which can be continuously optimized or changed, in |
| | contrast to “molecular” substances whose structural |
| | expression can only be changed via a variation in |
| | discrete states, for example that is by varying a |
| | substitution pattern. |
| Library of | This designates an arrangement comprising at least |
| materials: | two, that is a plurality, preferably at least 1 000, |
| | further preferably at least 100 000, in particular at |
| | least 10 000 000, and further preferably at least |
| | 1 000 000 000 substances or chemical compounds, |
| | mixtures of chemical compounds, formulations, |
| | which are termed “building blocks” in the context of |
| | the present invention. |
| Set: | The term “set” defines a composition of support |
| | bodies. |
| Performance | This term designates measurable characteristics of |
| characteristic: | the materials of the library of materials, such as |
| | catalytic activity or catalytic selectivity, which are |
| | determined using suitable sensors. Examples of |
| | these are given in the description. |
| Substance: | Chemical or biological entity, material, component, |
| | precursor or mixtures of two or more thereof. |
| Subset: | A “subset” is a collection of support bodies which is |
| | spatially separate from other subsets and obtained |
| | by dividing a set of support bodies, the number of |
| | support bodies in the subset always being less than |
| | the number of support bodies in the set. |
| Partial volume: | A “partial volume” is an amount of fluid obtained |
| | by dividing a volume of fluid that is less than the |
| | amount of fluid in the volume. |
| Support bodies: | This term comprises in principle all three- |
| | dimensional devices and bodies that have pores or |
| | boreholes or channels. The support body must be |
| | suitable to be brought in immediate and thorough |
| | contact with at least parts of at least one of the |
| | added substances. The term support body comprises |
| | pristine support bodies that are added to the library |
| | of materials as well as any support body during any |
| | step in the process of manufacturing a library of |
| | materials according to the invention, be it a |
| | contacting, conditioning, treatment or any other |
| | step. There are no restrictions with respect to the |
| | external shape of the support bodies, provided that it |
| | is a three-dimensional device or a three-dimensional |
| | body. Thus the support body can have the shape of a |
| | sphere or hollow sphere, an ellipsoidal body, a |
| | parallelepiped, a cube, a cylinder, a prism or a |
| | tetrahedron. The term “support body” therefore |
| | explicitly comprises three-dimensional devices for |
| | receiving fluids. |
| Subgroup: | A “subgroup” is a collection of support bodies |
| | which is not spatially separated from other |
| | subgroups and is obtained by dividing a set and/or |
| | subset of support bodies, the number of support |
| | bodies in the subgroup always being less than the |
| | number of support bodies in the set. |
| Subdivided | A “subdivided volume” is an amount of fluid which |
| volume: | is less than the amount of fluid in a partial volume, |
| | and which can be taken off from or added to a |
| | partial volume. |
| Mixing: | “Mixing” in the context of the invention, in addition |
| | to the mixing of at least two solutions, also means |
| | that the position of the support bodies within a |
| | vessel can be changed arbitrarily (local |
| | permutation). |
| Interchange: | “Interchange” in the context of the invention means |
| | spatial permutation of support bodies. |
| Volume: | The term “volume” means the totality of the fluid |
| | medium. |

Further advantages and aspects of the invention result from the description, the exemplary embodiments and the accompanying drawings.

Clearly, the abovementioned features and the features which are still to be described below can be used not only in the combination specified in each case, but also in other combinations or else alone, without departing from the context of the present invention.

The invention is illustrated diagrammatically in the drawings on the basis of exemplary embodiments and is described in detail below with reference to the drawings.

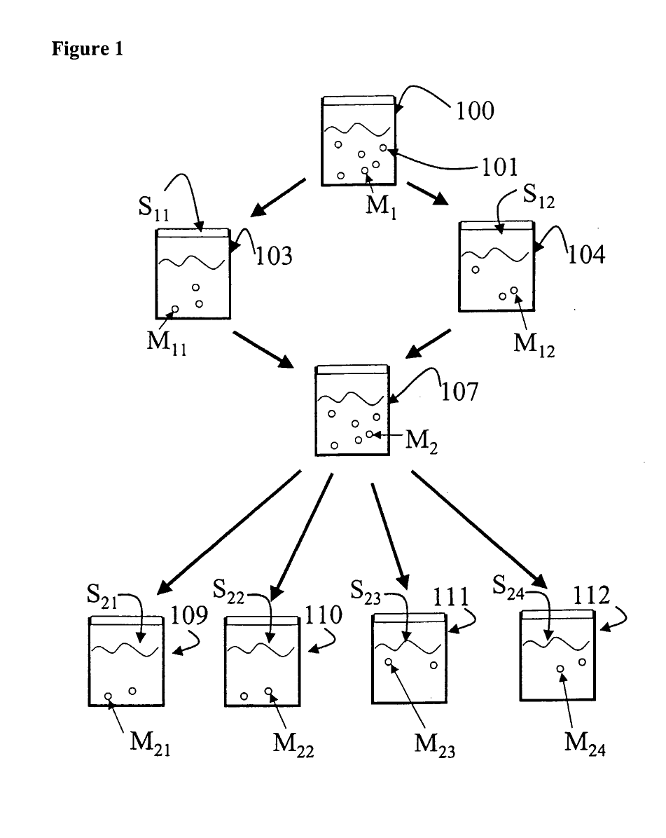

FIG. 1 shows diagrammatically the inventive process according to Claim 1.

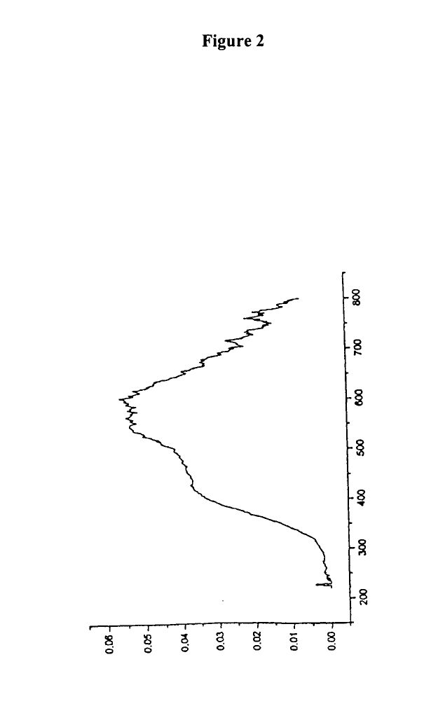

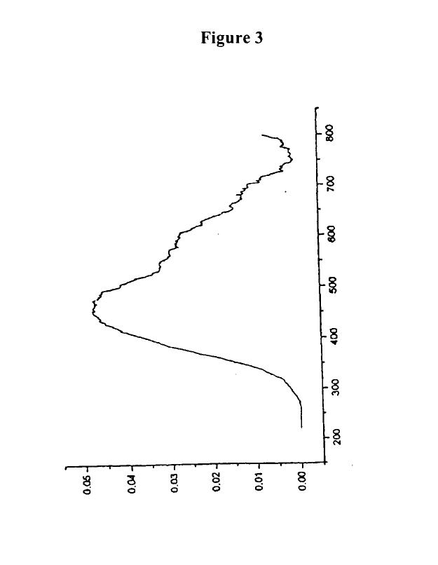

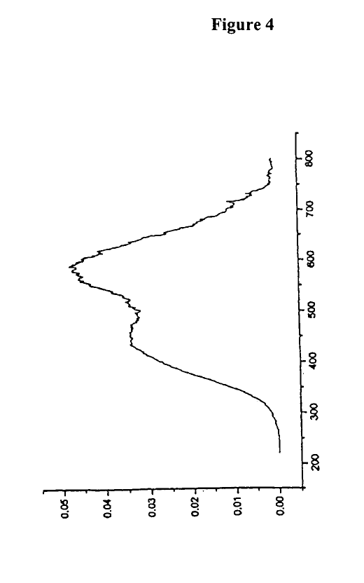

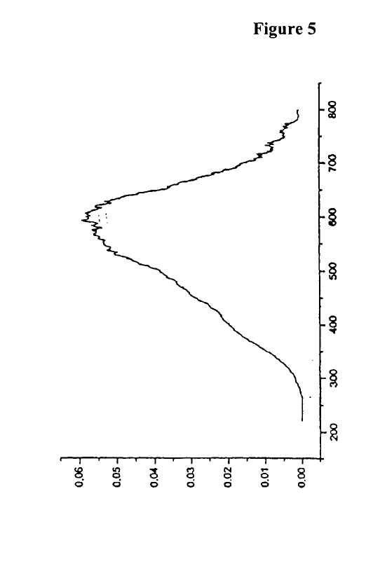

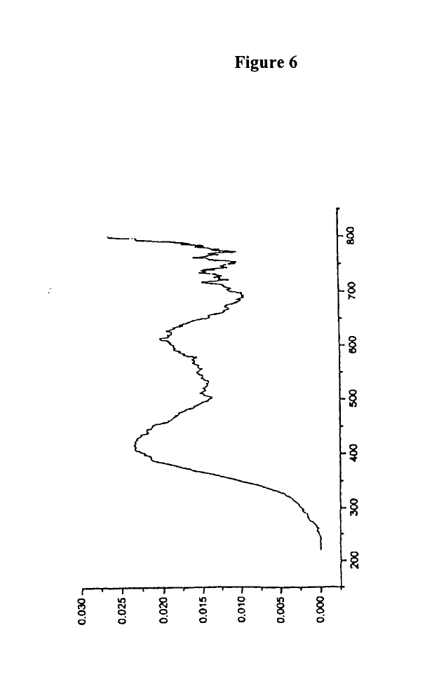

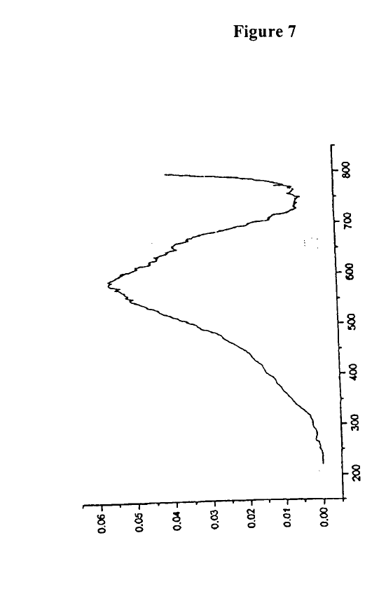

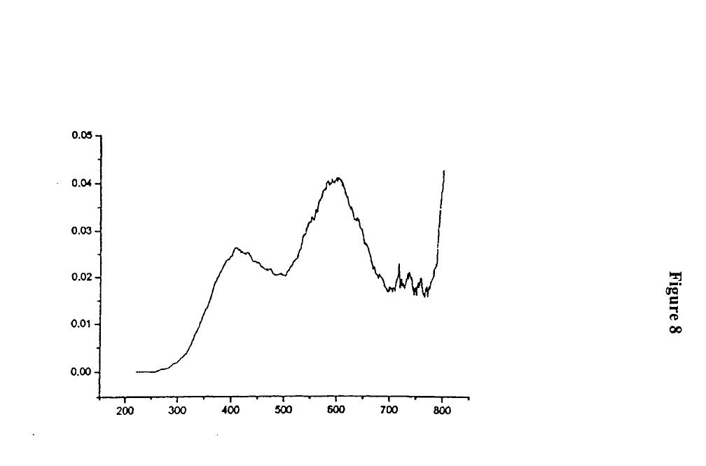

FIGS. 2 to 8 show analytical results carried out on building blocks of an inventive library of materials produced by the process according to Claim 1.

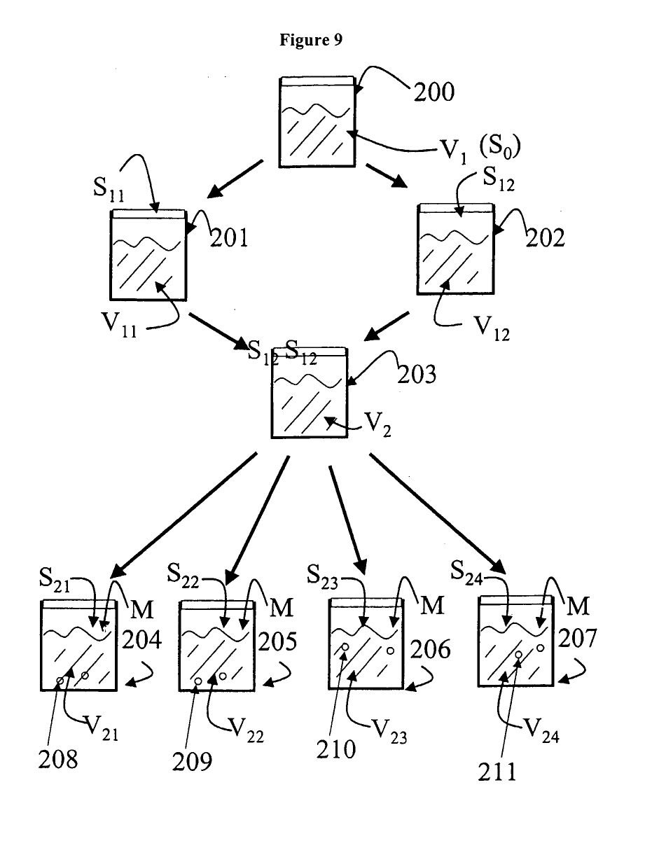

FIG. 9 shows the diagrammatic representation of the process according to Claim 16.

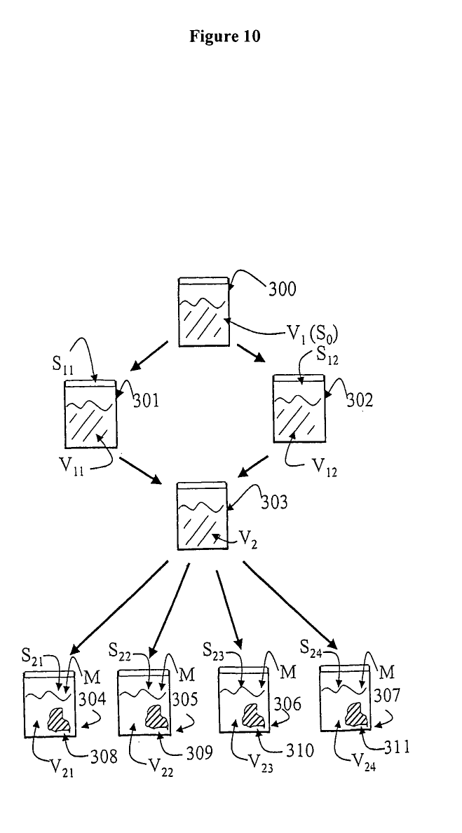

FIG. 10 shows a further embodiment of the process according to Claim 16.

FIG. 1 shows by way of example in a diagrammatic depiction the inventive process having the sequence of steps (0), (1), (2), (3), (1), (2). In a vessel 100 is situated a solution of a first substance 101 into which a set M1 of t₁, for example porous support bodies T1 of aluminum oxide, preferably beads, is introduced,

After contacting the set M1, the set M1 is divided into the first two subsets M₁₁ and M₁₂ which are distributed into containers 103 and 104 and are then contacted with solutions of substances S₁₂ and S₁₁.

After being contacted with the substances S₁₁ and S₁₂, the first two subsets M₁₁ and M₁₂ are combined to form a new set M₂ and, in the container 107, if appropriate, are contacted with a further substance which is not shown in FIG. 1. Also, obviously, further contacting with the substances S₁₁ and S₁₂ can be performed, or materials can be taken off from the sets M₁₁ and M₁₂.

The set M2 is then divided into four second subsets M₂₁, M₂₂, M₂₃ and M₂₄ which are contacted in vessels 109, 110, 111 and 112 with solutions of the substances S₂₁, S₂₂, S₂₃ and S₂₄. The subsets M₂₁ to M₂₄ are then isolated and the isolated support bodies contacted with the corresponding substances are subjected to a reactive treatment, for example a drying treatment or irradiation treatment.

FIG. 9 shows a further embodiment of the inventive process according to Claim 16. A volume V₁ in vessel 200 in which a substance S₀ was dissolved is transferred, in two partial volumes V₁₁ and V₁₂ into two vessels 201 and 202. Then, solutions of second substances S₁₁ and S₁₂ were added to the respective partial volumes V₁₁ and V₁₂. These partial volumes were then recombined to form a new volume V₂ in vessel 203. There was then a further division into further second partial volumes V₂₁ to V₂₄ in vessels 204 to 207. Further substances S₂₁ to S₂₄ were then added. After addition of the substances S₂₁ to S₂₄ agents M were added. These can be, as shown in FIG. 9, for example, also porous support beads of aluminum oxide 208, 209, 210 and 211. These were correspondingly impregnated with the respective multicomponent solutions formed by the resultant multicomponent systems in the vessels 204 to 207. These support beads 208 to 211 were then isolated and subjected to a thermal treatment of, for example, 80° C. for 15 h and were then tested for their catalytic characteristics.

FIG. 10 shows a further embodiment of the inventive process according to Claim 16. Similarly to FIG. 9, in a vessel 300, a volume V₁ in which a substance S₀ is dissolved was divided in a first step into the partial volumes V₁₁ and V₁₂ in two vessels 301 and 302. The substances S₁₁ and/or S₁₂ are then added in the solid state or in solution to the partial volumes V₁₁ and V₁₂. In a further step, the partial volumes V₁₁ and V₁₂ are then combined to form a second new volume V₂ in vessel 303. After being combined, the volume V₂ is divided into further partial volumes V₂₁ to V₂₄ in the vessels 304 to 307. Then substances or solutions of the substances S₂₁ to S₂₄ are added to the partial volumes V₁₂ to V₂₄. After addition is complete, an agent M is added to the solutions of vessels 304 to 307.

In contrast to FIG. 9, the agent M this time is a precipitation reagent or a conditioning reagent, so that the solution mixtures resulting in containers 304 to 307 precipitate out in the form of solids without support. Thus four different multicomponent systems 308 to 311 are obtained by precipitation. The precipitated substances can then further be subjected to a physicochemical secondary treatment. These multicomponent systems 308 to 311 can then be appropriately tested for their catalytic characteristics.

This may be described in more detail with reference to examples below.

### EXAMPLES

### Example 1

In Example 1, the following aqueous impregnation solutions of substances were used:

TABLE 1

| Substance | Concentration | Volume applied |
|---|---|---|
| Bi(NO₃)₃ | 1.25 M | 500 μl |
| Cu(NO₃)₂ | 4.6 M | 250 μl |
| Co(NO₃)₃ | 4 M | 250 μl |
| Mg(NO₃)₂ | 2.85 M | 500 μl |
| Fe(NO₃)₂ | 3 M | 250 μl |
| V₂(C₂H₄O₄)₅ | 0.45 M | 250 μl |
| HAuCl₄ | 0.22 M | 500 μl |

In a 1st step (1st generation), 500 μl of Bi solution were pipetted to 1 g of γ-aluminum oxide beads (obtained from CONDEA, 0.5 mm in diameter) evenly distributed over the surface in a porcelain dish. After intensive mixing, the set was halved and transferred to two new porcelain dishes; the first half of the beads was contacted with cobalt solution (=2nd generation Bi—Co), and the second half with copper solution (=2nd generation Bi—Cu). The two sets of the second generation were combined on a new dish, mixed and contacted with the magnesium solution (=3rd generation Bi—Co—Cu—Mg; not shown in Table 2). The mixed support bead set was then halved again and divided among two dishes; one half was contacted with a vanadium precursor solution (=4th generation Bi—Co—Cu—Mg—V), and the second with iron solution (=4th generation Bi—Co—Cu—Mg—Fe). Both sets were recombined, intensively mixed and in the last step were contacted with the gold solution (=final generation Bi—Co—Cu—Mg—Fe—V—Au).

Finally a drying step was carried out: the final generation was treated for 12 h at 80° C. in a drying cabinet. To monitor the result, some beads of each generation were taken off and studied by X-ray fluorescence analysis (μ-EDX). The results were summarized in Table 2 below. All data in per cent by weight (WT %) are based on the corresponding stable oxides of the metals. The third generation (Bi—Co—Cu—Mg) was not listed, since magnesium is not detectable as a light metal with the X-ray fluorescence analysis used.

A seven-component system ultimately results on aluminum oxide supports. At each synthesis step, as shown in Table 2, a number of beads were taken off and dried for 12 h at 80° C. Table 2 shows that all binary to heptary systems can be obtained in a simple manner. Each bead, considered statistically, has a different composition. Owing to the manner of liquid application, after each synthesis step there also exists a set of non-reacted beads, so that in addition to the desired heptary systems, all binary, ternary etc. component systems are also produced in parallel therewith. These subgroups can be specifically produced by taking out a bead set at a corresponding point in the production sequence.

The results are shown in Table 2 below:

TABLE 2
Results of the μ-EDX (micro X-ray fluorescence analysis
[micro-EDX]) on randomly selected support beads of
generations 1, 2, 4 and of the final generation from Example 1:

| | |
|---|---|
| Oxides: | % by weight |

1st generation: Bi

| | | |
|---|---|---|
| 1st bead | Al | 98.49 |
| | Bi | 1.4 |
| 2nd bead | Al | 99.07 |
| | Bi | 0.79 |

2nd generation: Bi—Co

| | | |
|---|---|---|
| 1st bead | Al | 96.85 |
| | Co | 3 |
| | Bi | 0.03 |
| 2nd bead | Al | 99.65 |
| | Co | 0.24 |
| | Bi | 0.02 |
| 3rd bead | Al | 98.05 |
| | Co | 1.83 |
| | Bi | 0.03 |
| 4th bead | Al | 98.12 |
| | Co | 0.6 |
| | Bi | 1.22 |
| 5th bead | Al | 97.59 |
| | Co | 1.59 |
| | Bi | 0.73 |

2nd generation: Bi—Cu

| | | |
|---|---|---|
| 1st bead | Al | 94.89 |
| | Cu | 4.97 |
| | Bi | 0.04 |
| 2nd bead | Al | 98.19 |
| | Cu | 1.02 |
| | Bi | 0.68 |
| 3rd bead | Al | 99.79 |
| | Cu | 0.04 |
| | Bi | 0.04 |
| 4th bead | Al | 99.75 |
| | Cu | 0.13 |
| | Bi | 0.06 |
| 5th bead | Al | 98.03 |
| | Cu | 1.13 |
| | Bi | 0.77 |

4th generation Bi—Co—Cu—Mg—Fe

| | | |
|---|---|---|
| 1st bead | Al | 94 |
| | Fe | 4.9 |
| | Co | 0.45 |
| | Cu | 0.47 |
| | Bi | 0.1 |
| 2nd bead | Al | 94.03 |
| | Fe | 1.39 |
| | Co | 0.77 |
| | Cu | 3.66 |
| | Bi | 0.1 |
| 3rd bead | Al | 93.9 |
| | Fe | 3.05 |
| | Co | 0.29 |
| | Cu | 2.57 |
| | Bi | 0.11 |
| 4th bead | Al | 95.31 |
| | Fe | 0.29 |
| | Co | 4.1 |
| | Cu | 0.19 |
| | Bi | 0.05 |
| 5th bead | Al | 97.58 |
| | Fe | 1.83 |
| | Co | 0.14 |
| | Cu | 0.34 |
| | Bi | 0.05 |

4th generation: Bi—Co—Cu—Mg—V

| | | |
|---|---|---|
| 1st bead | Al | 99.61 |
| | V | 0.08 |
| | Co | 0.04 |
| | Cu | 0.22 |
| | Bi | 0.02 |
| 2nd bead | Al | 96.48 |
| | V | 2.81 |
| | Co | 0.29 |
| | Cu | 0.36 |
| | Bi | 0.03 |
| 3rd bead | Al | 94.47 |
| | V | 3.89 |
| | Co | 1.23 |
| | Cu | 0.25 |
| | Bi | 0.07 |
| 4th bead | Al | 94.91 |
| | V | 2.45 |
| | Co | 2.22 |
| | Cu | 0.23 |
| | Bi | 0.14 |
| 5th bead | Al | 92.11 |
| | V | 4.69 |
| | Co | 0.52 |
| | Cu | 1.18 |
| | Bi | 1.46 |
| | Co | 1.67 |
| | Cu | 1.06 |
| | Au | 0.37 |
| | Bi | 0.74 |

Final generation: Bi—Cu—Co—Mg—Fe—V—Au

| | | |
|---|---|---|
| 1st bead | Al | 93.83 |
| | V | 0.5 |
| | Fe | 1.83 |
| | Co | 1.67 |
| | Cu | 1.06 |
| | Au | 0.37 |
| | Bi | 0.74 |
| 2nd bead | Al | 92.59 |
| | V | 0.53 |
| | Fe | 0.44 |
| | Co | 0.43 |
| | Cu | 1.8 |
| | Au | 0.24 |
| | Bi | 3.98 |
| 3rd bead | Al | 96.39 |
| | V | 0.19 |
| | Fe | 2.43 |
| | Co | 0.2 |
| | Cu | 0.6 |
| | Au | 0.04 |
| | Bi | 0.15 |
| 4th bead | Al | 97.75 |
| | V | 0.35 |
| | Fe | 0.3 |
| | Co | 0.21 |
| | Cu | 1.16 |
| | Au | 0.11 |
| | Bi | 0.13 |
| 5th bead | Al | 94.87 |
| | V | 0.19 |
| | Fe | 2.71 |
| | Co | 0.63 |
| | Cu | 1.23 |
| | Au | 0.15 |
| | Bi | 0.23 |
| 6th bead | Al | 97.28 |
| | V | 0.2 |
| | Fe | 0.36 |
| | Co | 0.23 |
| | Cu | 1 |
| | Au | 0.04 |
| | Bi | 0.88 |
| 7th bead | Al | 98.17 |
| | V | 0.1 |
| | Fe | 1.04 |
| | Co | 0.16 |
| | Cu | 0.44 |
| | Au | 0.01 |
| | Bi | 0.09 |
| 8th bead | Al | 94.98 |
| | V | 1.65 |
| | Fe | 0.47 |
| | Co | 0.65 |
| | Cu | 2.05 |
| | Au | 0.08 |
| | Bi | 0.11 |
| 9th bead | Al | 98.58 |
| | V | 0.14 |
| | Fe | 0.41 |
| | Co | 0.16 |
| | Cu | 0.39 |
| | Au | 0.05 |
| | Bi | 0.27 |
| 10th bead | Al | 97.71 |
| | V | 0.33 |
| | Fe | 0.61 |
| | Co | 0.34 |
| | Cu | 0.6 |
| | Au | 0.22 |
| | Bi | 0.19 |

### Example 2

### Example 2

In the first step, a starting set of 3.5 g of porous y-aluminum oxide (bead diameter 0.5 mm, obtained from Condea) was divided into 7 equal sets (each of 0.5 g) and distributed among 7 dishes (split). The sets in the dishes were given the designations F′, G′, H′, J′, K′, L′ and M′.

The impregnation solutions used were aqueous solutions of nitrates of the following metals: Zn, Fe, Ni, Cu, Co, Mg and Ca. The concentration of the respective solutions was 2.5 mol/l for all solutions, based on the respective metal. 200 μl of a metal salt solution were pipetted in each case onto the sets F′, G′, H′, J′, K′, L′ and M′:

TABLE 3

| | | | | | | | |
|---|---|---|---|---|---|---|---|
| Set | F’ | G’ | H’ | J’ | K’ | L’ | M’ |
| Metal salt | Zn | Fe | Ni | Cu | Co | Mg | Ca |
| solution | | | | | | | |

The sets F′, G′, H′, J′, K′, L′ and M′ were then dried at 80° C. for 2 h and combined to form a set 1 (mix). The set 1 was then divided into 7 equal sets F″, G″, H″, J″, K″, L″ and M″ (split).

200 μl of a further metal salt solution were pipetted in each case onto the sets F″, G″, H″, J″, K″, L″ and M″:

TABLE 4

| | | | | | | | |
|---|---|---|---|---|---|---|---|
| Set | F’ | G’ | H’ | J’ | K’ | L’ | M’ |
| Metal salt | Zn | Fe | Ni | Cu | Co | Mg | Ca |
| solution | | | | | | | |

The sets F″, G″, H″, J″, K″, L″ and M″ were then dried at 80° C. for 2 h and combined to form a set 2 (mix). The set 2 was then divided into 7 equal sets F, G, H, J, K, L and M (split).

200 μl of a further metal salt solution were pipetted in each case onto the sets F, G, H, J, K, L and M:

TABLE 5

| | | | | | | | |
|---|---|---|---|---|---|---|---|
| Set | F | G | H | J | K | L | M |
| Metal salt | Zn | Fe | Ni | Cu | Co | Mg | Ca |
| solution | | | | | | | |

The sets F, G, H, J, K, L and M were then dried at 80° C. for 2 h and then calcined at 1 000° C. for 5 h in order to produce compounds of the spinel type on the aluminum oxide beads.

Individual beads were selected from the sets F, G, H, J, K, L and M and subjected to UV-Vis spectroscopic analysis in diffuse reflection. The following figures show the spectra obtained for a number of individual beads from the respective sets. By way of example, spectra form the beads from set F are shown in FIGS. 2 to 8. In this case compounds were obtained of the mixed oxide type, for example spinels, perowskites, elpasolites, etc., which can be used as color pigments.

### Example 3

The aqueous impregnation solutions were the same as the ones used in Example 1 (see Table 1).

In a 1st step (1st generation), 500 μl of Bi solution were pipetted to 1 g of γ-aluminum oxide beads (obtained from CONDEA, 0.5 mm in diameter) evenly distributed over the surface in a porcelain dish. After intensive mixing, the set was halved and transferred to two new porcelain dishes; the first half of the beads was contacted with cobalt solution (=2nd generation Bi—Co), and the second half with copper solution (=2nd generation Bi—Cu). The two sets of the second generation were not combined as in Example 1 but contacted with the magnesium solution (=3rd generation Bi—Co—Mg and Bi—Cu—Mg). Since the “pooling” step is omitted, this example is termed the “direct divide” case. The mixed support bead sets were then halved again and divided among two dishes; one half was contacted with a vanadium precursor solution (=4th generation Bi—Co—Cu—Mg—V), and the second with iron solution (=4th generation Bi—Co—Cu—Mg—Fe). Both sets were recombined, intensively mixed and in the last step were contacted with the gold solution (=final generation Bi—Co—Cu—Mg—Fe—V—Au).

Finally a drying step was carried out: the final generation was treated for 12 h at 80° C. in a drying cabinet. To monitor the result, some beads of each generation were taken off and studied by X-ray fluorescence analysis (μ-EDX). The results were summarized in Table 2. All data in per cent by weight (WT %) are based on the corresponding stable oxides of the metals. The third generation (Bi—Co—Cu—Mg) was not listed, since magnesium is not detectable as a light metal with the X-ray fluorescence analysis used.

A seven-component system ultimately results on aluminum oxide supports. At each synthesis step, as shown in Table 2, a number of beads were taken off and dried for 12 h at 80° C. Table 2 shows that all binary to heptary systems can be obtained in a simple manner. Each bead, considered statistically, has a different composition. Owing to the manner of liquid application, after each synthesis step there also exists a set of non-reacted beads, so that in addition to the desired heptary systems, all binary, ternary etc. component systems are also produced in parallel therewith. These subgroups can be specifically produced by taking out a bead set at a corresponding point in the production sequence.

FIG. 2 through FIG. 8 show representative results of UV-VIS spectroscopic measurements on selected individual beads from set F. As can be seen from the spectra each of the seven studied beads has a different composition of the spinel-type compound present on it. The same results, i.e. different compositions on different beads have been found for individual beads from other sets, such as the sets G, H, J, K, L or M. For all spectra, the x-axis represents the wavelength given in nanometers and the y-axis represents F(R) in Kubelka-Munk units.

1. Process for producing a multiplicity of building blocks of a library of materials comprising a sequence of the following steps:
(2) contacting all or parts of a number t₁ₘ of support bodies of the kind T1 that are part of at least one subset M₁ₘ with at least one first substance of the kind S₁ₛ, characterized in that the support bodies are porous and that at least parts of the substance penetrate into at least parts of the bulk of the support bodies, and where s, t₁ and m are mutually independent integers ≧1;
(3) combining parts of or complete subsets M₁ₘ to result in one or more second sets M₂ₙcontaining a number t₂ₙ, of support bodies of the kind T1, where n, m and t₂ are integers ≧1 and n<m.

2. Process for producing a multiplicity of building blocks of a library of materials according to claim 1, which additionally comprises the following step:
(1) dividing a first set M₁ containing a number t₁ of support bodies of the kind T1 into a number k of first subsets M₁ₘ containing a number t₁ₘ of support bodies, where m and t₁ are mutually independent integers ≧1, and t₁≧k with k being an integer ≧2.

3. Process according to claim 1 or 2, comprising the following step:
(0) contacting a first set M₁ containing a number t₁ of support bodies of the kind T1 with at least one substance zero of the kind S₀ₛ, where s has the meaning as in claim 1.

4. Process for producing a multiplicity of building blocks of a library of materials, comprising a sequence of the following steps:
(I) contacting of at least one first substance of the kind S₁ₛ with a first set M₁ containing a number t₁ of support bodies of the kind T1;
(1) dividing a first set M₁ containing a number t₁ of support bodies of the kind T1 into k first subsets M₁ₘ containing a number t₁ₘ of support bodies of the kind T1, where m and t₁ are mutually independent integers ≧1, and t₁≧k with k being an integer ≧2.

5. Process according to claim 4 which comprises the sequence of the following steps:
(2)-(3)-(I)-(1);
(3)-(2)-(I)-(1);
(2)-(I)-(3)-(1); or
(3)-(1)-(I)-(2).

6. Process according to any of the preceding claims, in which the first set M₁ containing a number t₁ of support bodies comprises at least two different support bodies of the kind T1X, where X is an integer ≧1.

7. Process according to any of the preceding claims, in which building blocks and/or identical or different support bodies of the kind T2Y which are identical to or different from the support bodies of the kind T1 or T1X, where Y is an integer ≧1, are added to at least one of the subsets M₁ₘ before and/or after and/or during the respective steps (I), (0), (1), (2) or (3) or the totality of the steps or a sequence of the steps defined in claims 1 to 5.

8. Process according to any of the preceding claims, in which a freely selectable number of building blocks and/or support bodies T1 and/or T1X and/or T2Y can be taken off from and/or added to at least one of the subsets M₁ₘ before and/or after and/or during the steps defined in claims 1 to 5.

9. Process according to any of the preceding claims, in which before and/or after and/or during and/or instead of at least one of the steps (I) and (0) to (3) at least one physical and/or chemical and/or physicochemical treatment is carried out on at least one of the support bodies, in which case the respective treatment of any of the individual support bodies can be identical or different.

10. Process according to any of the preceding claims comprising a sequence of the following steps:
(1′) contacting a first subgroup of sets and/or subsets of support bodies with at least one first zero substance S₀ₛ and/or at least one first substance S₁ₛ, with s being identical or different for each time this step is repeated;
(2′) mixing with one another respective sets and/or subsets contacted according to (1′), and
(3′)contacting a second subgroup of sets or subsets of support bodies with at least one second substance S₂ₛ which is identical to or different from the substances S₁ₛ and/or S₀ₛ with s being identical or different for each time this step is repeated.

11. Process according to claim 10, in which support bodies of the first and/or second subgroup are contacted with at least one substance using a local spray system or a local spray system with mask or diaphragm to cover the remaining support bodies.

12. Process according to any of the preceding claims, in which the subsets are the same size or of different sizes.

13. Process according to any of the preceding claims, characterized in that the substances are contacted in accordance with a procedure which is selected from: addition of solution, suspension, dispersion, emulsion, melt or from powder-coating processes.

14. Process according to any of the preceding claims, characterized in that at least two of the substances react in any possible combination with one another or the support body or both after at least one contacting step.

15. Process according to any of the preceding claims, in which the sequences of steps defined therein are permuted and/or repeated a number of times, and where the substances S₀ₛ, S₁ₛ and/or S₂ₛ can be the same as or different from the respective substances used in any of the previous steps and/or where the subsets M₁ₘ and/or M₂ₙ can be the same as or different from the respective subsets formed in any of the previous steps and where the numbers and kinds of support bodies contacted with the substances and/or added or taken from any (sub)set can be the same as or different from any of the previous steps.

16. Process according to any of the preceding claims, characterized in that at least one of the support bodies used in any of the steps or added to and/or withdrawn from any of the (sub)sets can be altered due to at least one physical or chemical or physicochemical treatment or any combination thereof.

17. Process for producing a multiplicity of building blocks of a library of materials comprising a sequence of the following steps:
(2) introducing at least one first substance S₁ₛ into all or into some of a number k of partial volumes V₁ₘ, where m and s are integers ≧1 and k is an integer ≧2;
(3) combining parts of the partial volumes, or complete partial volumes V₁ₘ resulting in a plurality of second volumes V₂ₙ, where n is an integer and n<m.

18. Process according to claim 17 which comprises the following additional step:
(1) dividing a volume V₁ of a fluid medium FM₁ into a number k of first partial volumes V₁ₘ, where m is an integer ≧1 and k is an integer ≧2.

19. Process according to claim 17 or 18, comprising the following step (0):
(0) combining the first volume V₁ with at least one zero substance S₀ₛ, where s has the meaning as in claim 17.

20. Process for producing a multiplicity of building blocks of a library of materials comprising a sequence of the following steps:
(I) introducing at least one first substance S₁ₛ into a first volume V₁;
(1) dividing a first volume V₁ into a number k of first partial volumes V₁ₘ, where m is an integer ≧1 and k is an integer ≧2.

21. Process according to any of claims 17 to 20 which comprises a sequence of the following steps:
(2)-(3)-(I)-(1);
(3)-(2)-(I)-(1);
(2)-(I)-(3)-(1); or
(3)-(1)-(I)-(2).

22. Process according to any of claims 17 to 21, in which a freely selectable number of subdivided volumes UV₁ₘ is taken off from and/or added to at least one of the partial volumes V₁ₘ before and/or after and/or during the respective steps (I), (0), (1), (2) or (3) or the totality of the steps or sequence of steps defined in claims 17 to 20, and m has the meaning as defined in claim 17.

23. Process according to any of claims 17 to 22, in which before and/or after and/or during and/or instead of steps (I) and (0) to (3) at least one physical and/or chemical and/or physicochemical treatment is carried out on at least one of the partial volumes V₁ₘ and/or subdivided volumes UV₁ₘ, in which case the respective treatment of the individual partial volumes and/or subdivided volumes can be identical or different.

24. Process according to any of claims 17 to 23, comprising a sequence of the following steps:
(1′) introducing at least one first substance zero S₀ₛ and/or at least one first substance S₁ₛ into a first partial volume V₁ₘ and/or subdivided volume UV₁ₘ;
(2′) interchanging with one another the respective partial volume and/or subdivided volume obtained according to (1′) and
(3′) introducing at least one second substance S₂ₛ which is identical to or different from the substances S₁ₛ and/or S₀ₛ into a second subdivided volume of the volumes or partial volumes.

25. Process according to any of claims 17 to 24, characterized in that an agent is added to the partial volumes V₁ₘ and/or subdivided volumes UV₁ₘ, which agent enables at least a part of the dissolved substances to be obtained in solid form.

26. Process according to claim 25, characterized in that said agent is a precipitation reagent.

27. Process according to any of claims 17 to 26, characterized in that the partial volumes and/or subdivided volumes are the same size or different sizes.

28. Process according to any of the preceding claims 17 to 27, characterized in that at least two of the substances react with each other in any possible combination after at least one contacting step.

29. Process according to any of claims 17 to 28, characterized in that the sequences of steps defined therein are permuted and/or repeated in analogous or equivalent ways any given number of times.

30. Process according to any of claims 17 to 29, characterized in that the precipitated substances are subjected to a physical and/or chemical and/or physicochemical treatment in which case the respective treatment of any of the individual partial volumes or precipitated or otherwise formed bodies can be identical or different.

31. Library of materials obtainable by a process according to any of the preceding claims.

32. Process according to any of the preceding claims for identifying different building blocks of a library of materials, characterized in that each building block is provided with an individualizable coding.

33. Process according to claim 32, characterized in that the coding is substantially chemically inert

34. Process according to claim 33, characterized in that the coding is sufficiently inert to remain readable by a designated device even after having been subjected to all steps or treatments as defined by the inventive process.

35. Process according to any of claims 31 to 34, characterized in that the coding can be read by analytical techniques selected from RFA, UV/VIS spectroscopy, detection of high-energy radiation, NMR spectroscopy, ESR spectroscopy, Mössbauer spectroscopy, SIMS, optical methods (fluorescence spectroscopy, phosphorescence spectroscopy).

36. Process for determining at least one performance characteristic and/or property of building blocks in a library of materials according to any of the preceding claims, characterized in that the process comprises the following step:
(a) determination of at least one first performance characteristic and/or property of at least one building block using at least one sensor.

37. Process according to claim 36 which additionally comprises the following step (b):
(b) determination of at least one further performance characteristic and/or property of the at least one building block using at least one further sensor.

38. Process according to claim 36 or 37, characterized in that the further performance characteristic and/or property is only determined for a selected group of building blocks.

39. Process according to claim 38, characterized in that the selection of the building blocks that lead to determining the further performance characteristic and/or property depends on the result of the determination of the first performance characteristic and/or property.

40. Process according to any of claims 36 to 39, characterized in that the building blocks are selected for the further steps according to the invention automatically by a data processing system.

41. Process according to any of claims 36 to 40, characterized in that, before step (b), at least one starting material is introduced at at least two building blocks of the library of materials, which are separated from one another, to carry out a chemical and/or physical and/or physicochemical step in the presence of at least one building block and, after it flows through or around the building block, an effluent stream is obtained.

42. Process according to any of claims 36 to 41, characterized in that the sensor is based on a method for determination which is selected from the group consisting of infrared thermography, infrared thermography in combination with mass spectrometry, mass spectrometery, GC, LC, HPLC, micro-GC, dispersive FTIR spectroscopy, Raman spectroscopy, NIR spectroscopy, UV spectroscopy, UV/VIS spectroscopy, NMR spectroscopy, ESR spectroscopy, microwave spectroscopy, GC-MS spectrometry, infrared thermography/Raman spectroscopy, infrared thermography/dispersive FTIR spectroscopy, color detection with a chemical indicator/MS, color detection with a chemical indicator/GCMS, color detection with a chemical indicator/dispersive FTIR spectroscopy, photoacoustic analysis and tomographic NMR and ESR methods, electrochemical or chemical sensors.

43. Computer program containing program code means for carrying out the process according to one of the preceding claims.

44. Data carrier containing computer program according to claim 43.
